(19) United States
(12) Patent Application Publication
(10) Pub. No.: US 2009/0234252 A1
(43) Pub. Date: Sep. 17, 2009

(54) PORTABLE TOPICAL PAIN RELIEF SYSTEM

(75) Inventor: Eilaz Babaev, Minnetonka, MN (US)

Correspondence Address: Bacoustics, LLC, 5929 BAKER ROAD, SUITE 470, MINNETONKA, MN, 55345, US

(73) Assignee: Bacoustics, LLC, Minnetonka, MN (US)

(21) Appl. No.: 12/465,736

(22) Filed: May 14, 2009

Related U.S. Application Data
(63) Continuation-in-part of application No. 11/409,818, filed on Apr. 24, 2006.

(51) Int. Cl. A61N 7/00

(52) U.S. Cl. 601/2

(57) ABSTRACT

The invention discloses methods and devices to energize a portable carrier media which may be applied to a person's skin to provide pain relief. The carrier media may be recharged at an energizing station that ultrasonically activates the carrier media, such as a credit card, so that the plastic is energized by the exposure to the ultrasound into an activated state. The credit card can then held against the user's body to provide localized pain relief. If desired, the energized carrier media may be stored until needed in a suitable storage container such as a plastic bag or envelope. The energized carrier media is preferably held against the user's body with a suitable media attachment mechanism such as adhesive, tape or an elastic strap, however, the carrier media may be simply hand held in place for short periods of time.

### CROSS REFERENCE TO RELATED APPLICATIONS

This application is a continuation-in-part of U.S. patent application Ser. No. 11/409,818 filed Apr. 24, 2006.

### BACKGROUND OF THE INVENTION

The present invention relates to topical pain relief. More particularly, the present invention discloses apparatus and methods teaching a pain relief system that includes an energizing station and a carrier media such as a credit card that is portable, convenient to store and use and may provide portable topical pain relief when applied to a portion of a body.

Pain, often but not exclusively associated with injury, trauma, arthritis, muscle soreness and headache may be treated with various forms of energy. Most often the energy chosen is a variant of thermal energy, which in particular is heat or cold applied via a portable pad or pack. Applying thermal energy with a portable pack or pad is generally accomplished by means of a chemical reaction or energy transfer by placing the pad or pack in hot environment, such as boiling water or a microwave oven, or a cold environment, such as using ice, a refrigerator or freezer.

Applying chemicals and creams to the affected area and allowing them to evaporate can generate a local cooling effect to a portion of a user's body experiencing persistent lingering pain. The use of creams and chemicals is disadvantaged by the fact that such creams and chemicals are often messy to apply and can cause irritation to some, particularly if they come in contact with the user's eyes or mucosal membranes.

### SUMMARY OF THE INVENTION

The present invention is directed towards apparatus and methods for pain relief by a carrier media such as a credit card that is energized by exposure to ultrasound into an activated state so that placing the carrier media against the user's body may provide localized pain relief. The energized carrier media may be stored until needed in a suitable storage container to maintain the activated state. The energized carrier media is preferably held against the user's body with a suitable media attachment mechanism, however, the carrier media may be simply hand held in place for short periods of time.

The energizing station may be designed to utilize ultrasound energy to activate the carrier media. Other alternative activation energies include electrical, magnetic or electrostatic energies. In an ultrasound embodiment, the energizing station would typically include a credit card reader equipped to intake and activate the user's card or a kiosk dispenser to activate and dispense the carrier media. The energizing station may be designed specifically for energizing the media, or it may be of a multi-functional design. Compatible multi-function designs include credit/debit card readers, ATM's, food/beverage vending machines, CD/DVD dispensers and automated pharmacy dispensers.

An energizing station utilizing ultrasound energy for activation would typically require a power supply powering an ultrasound generator. The ultrasound generator would provide a driving signal to generate mechanical vibrations within an ultrasound transducer. These vibrations would be transmitted through an ultrasound horn having an ultrasound tip. Ultrasound waves emitted through the tip are preferably applied to the carrier media either by directly contacting the ultrasound tip or using a coupling medium between the ultrasound tip and the carrier media to enhance the efficiency of the ultrasound transmissions.

The carrier media may be of a reusable or of a disposable (one use) design. The carrier media may be specifically designed and manufactured for this application. In addition, existing products already commercially available may be utilized with the invention. Embodiments of carrier media include plastic films, credit/debit cards, adhesive bandage, or blank plastic cards. The present invention may be combined with an Acu-Node™ pliable pad to provide short-term pain relief to enhance and complement the longer-term treatment of chronic pain through the Acu-Node™ system.

The carrier media is preferably of an inexpensive plastic construction particularly in a disposable embodiment. Carrier media materials of construction may include; PVC (polyvinylchloride), PVCA (polyvinylchloride acetate), LDPE (low density polyethylene), PE and latex rubber.

Exposing a carrier media such as a credit card to ultrasonic waves energizes the plastic in the credit card which can then be used to provide pain relief. The energized plastic may be applied to a user to provide an analgesic effect either immediately after being energized or the energized carrier media can be stored for use at a future time.

If not used immediately, the carrier media is preferable placed within a storage container to avoid dissipation of the activation energy. The storage container may be a simple polyethylene envelope or a rigid case constructed of a plastic or metal material.

The carrier media may be simply held in place by the user, or for the user's convenience, a media attachment mechanism may be used. An adhesive bandage would often include its own adhesive for attachment to the user's skin. Alternatively, an elastic or Velcro strap may be used to hold the carrier media in place. In addition, an adhesive film or tape may be used to hold the carrier media in place.

This invention provides a very flexible system to provide convenient access to a pain relief system that is safe, inexpensive, simple to use and does not require administration or control of chemicals or drugs. The carrier media may be made available in a form that is easy to use and provides a very convenient portable device that can be activated at home for routine users, or at convenient locations for occasional users.

### BRIEF DESCRIPTION OF THE DRAWINGS

The present invention will be shown and described with reference to the drawings of preferred embodiments and clearly understood in details.

FIG. 1 is an elevational view of the energizing station incorporated into a kiosk or ATM.

FIG. 2 is a schematic representation of several components of the energizing station.

FIGS. 3A and 3B are representations of alternative embodiment of the invention.

FIG. 4 is a representation of an alternative embodiment of the invention showing the ultrasound wave reflection from a base material.

FIG. 5 is a representational view of an energizing station having a rotating ultrasound apparatus that can energize moving polymers from the radial side of an ultrasound tip

FIG. 6 is a cross-sectional view of an energizing station with a rotating ultrasound tip capable of energizing moving polymers from the radial side of the ultrasound tip

FIG. 7 is a perspective view of a carrier media within a storage container.

FIG. 8 is a perspective schematic view of a carrier media held in place on a user with a media attachment mechanism.

FIG. 9 is a perspective schematic view of a carrier media held in place on a user.

### DETAILED DESCRIPTION OF THE INVENTION

The present invention is directed towards apparatus and methods for pain relief. The invention may include an energizing station that ultrasonically activates a carrier media such as a credit card such that the plastic in the carrier media is energized by the exposure to the ultrasound into an activated state. The credit card can then held against the user's body to provide localized pain relief. If desired, the energized carrier media may be stored until needed in a suitable storage container. The energized carrier media is preferably held against the user's body with a suitable media attachment mechanism such as adhesive, tape or an elastic strap, however, the carrier media may be simply hand held in place for short periods of time.

The energizing station may be designed to utilize ultrasound energy to activate the carrier media. Other alternative activation energies include electrical, magnetic, electrostatic, microwave, laser, EMF and radio frequency radiation. In an ultrasound embodiment, the energizing station would typically include a credit card reader to intake and activate the user's card or a kiosk dispenser to activate and dispense the carrier media.

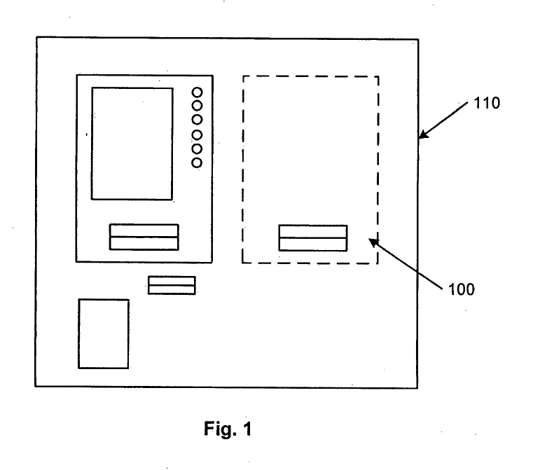

The energizing station may be designed specifically for energizing the media for in-home, drug stores or medical facilities. Alternatively it may be of a multi-functional design compatible with other uses. Examples of multi-function designs include credit/debit card readers, ATM's, vending machines, CD/DVD dispensers and automated pharmacy dispensers. FIG. 1 shows a view of the energizing station 100 incorporated into a kiosk 110 such as an ATM.

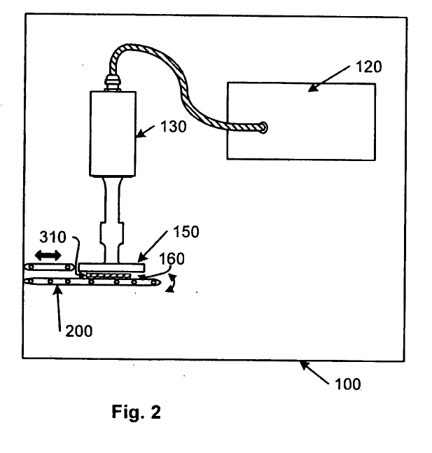

FIG. 2 shows a schematic representation of several components of the energizing station 100 in an embodiment not including a mechanism to intake the carrier media 310. This embodiment would have a less automated activation step, but may provide more flexibility with respect to types of media and control of activation parameters.

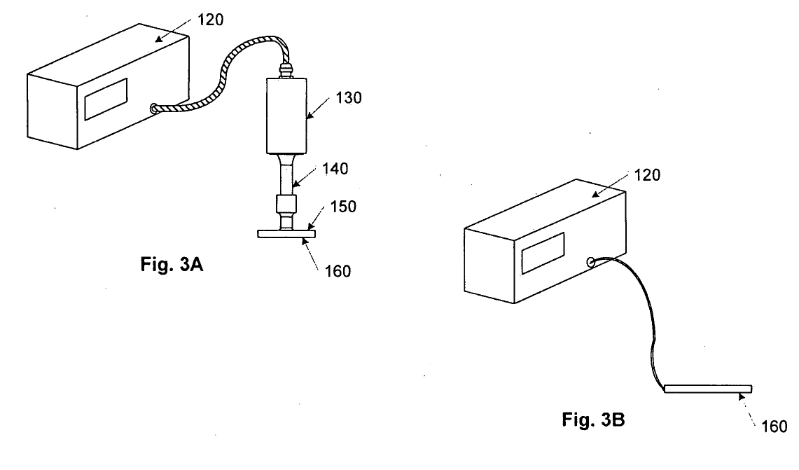

FIGS. 3A and 3B are representations of an alternative embodiment of the invention wherein the major components are not enclosed in a cabinet.

With regard to FIG. 3A, an energizing station utilizing ultrasound energy for activation would typically require a power supply powering an ultrasound generator 120. The ultrasound generator 120 would provide an electrical driving signal to generate mechanical vibrations within an ultrasound transducer 130. These vibrations would be transmitted through an ultrasound horn 140 having an ultrasound tip 150. The ultrasound horn may include the ultrasound tip 150 as a single unit, or the ultrasound tip 150 may be removeably attached through threads, mechanical fasteners, or other means.

At or near the distal end of the ultrasound tip 150 is the radiation surface 160 from which the ultrasound waves emitted through the ultrasound tip 150 may be applied to the carrier media 310 through the radiation surface 160 of the ultrasound tip 150 to the carrier media 310. Preferably the ultrasound waves are applied to the carrier media 310 either by directly contacting the radiation surface 160 of the ultrasound tip 150 or using a coupling medium between the radiation surface 160 of the ultrasound tip 150 and the carrier media 310 to optimize the efficiency of the ultrasound transmissions. The radiation surface 160 may be configured to emit ultrasound energy either radially or longitudinally from the ultrasound tip 150.

With regard to FIG. 3B, the ultrasound generator is electrically connected to a piezo-material for converting the electrical energy directly into ultrasound energy at the radiation surface 160. Use of certain piezo material which can be produced in a flat plate configuration, may be advantageous in that the radiation surface 160 may be easily constructed in the form of flat plates. This may be advantageous for providing flexibility in design of energizing station 100 of the carrier media 310 so that the radiation surface 160 can mirror the shape of the carrier media 310.

A card feeder 200 may be used to move the carrier media 310 to the radiation surface 160. Ultrasonic waves may then be delivered to the carrier media 310 through direct contact, a coupling medium, or without contacting the carrier media 310. Direct contact indicates that at least a portion of the ultrasound tip 150 delivering ultrasonic waves physically contacts at least a portion of the carrier media 310.

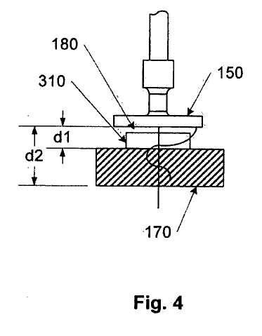

FIG. 4 is a representation of an alternative embodiment of the invention showing the ultrasound wave reflection from a base material 170. It may be preferable to ultrasonically energize a polymer while it is located on a base material such as, but not limited to metals, plastics, ceramics, rubbers, fabrics, composite materials, or any other similarly effective base materials or a combination thereof. Also represented is transmission of the ultrasonic waves delivered to a carrier media 310 through a coupling medium 180 such as, but not limited to, gel, liquid, or a polymer.

The ultrasonic waves may be generated having a frequency between 15 kHz and 40 MHz with a preferred frequency range of approximately 20 kHz-approximately 40 kHz. The recommended low-frequency ultrasound value is approximately 30 kHz and the recommended high-frequency ultrasound value is approximately 3 MHz.

The amplitude of the ultrasound waves can be 1 micron and above. The preferred amplitude range for low-frequency ultrasound is approximately 50 microns to approximately 60 microns, and the recommended amplitude value for low-frequency ultrasound is approximately 50 microns.

In one embodiment, the ultrasound waves may be delivered with an intensity of at least 0.001 watts/cm² for at least 0.1 seconds.

The time of sonication will vary based on factors such as the ultrasound frequency, amplitude, intensity, the type of carrier media 310, the thickness of carrier media 310, the type of base material, the thickness of base material. Sonication times within the range of 1 to 60 seconds are preferred.

For example, one could generate ultrasonic waves with a frequency of 30 kHz and amplitude of 60 microns and deliver these ultrasonic waves to a crystalline polymer for about 30 seconds, thereby energizing the polymer.

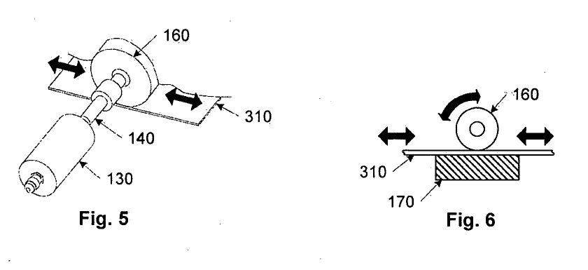

FIG. 5 is a representational view of an energizing station having a rotating ultrasound tip 150 that can energize a carrier media 310 such as a credit card moving along the radial side of an ultrasound tip 150, the radial side of the ultrasound tip 150 having a radiation surface 160. With this embodiment, the radiation surface 160 can be contacted with the carrier media 310 and may be energized as it moves past the radiation surface 160 without creating undo friction between the surfaces and the carrier media 310.

FIG. 6 is a cross-sectional view of an energizing station with a rotating ultrasound tip 150 capable of energizing moving carrier media 310 using a base material with a reflective surface (e.g. metal) during sonication. This may allow the ultrasonic waves to reflect off the base material back toward the carrier media 310 and thereby cause both sides of the base material to be “multiplicatively exposed” to ultrasonic waves.

As also shown in FIG. 6, the amount of reflection may vary depending on the distance d₁ between the ultrasound tip 150 and the lower surface level of the carrier media 310 and may also vary depending on the distance d₂ between the ultrasound tip 150 and the lower surface level of the base material.

The radiation surface 160 of the ultrasound tip 150 whether in an end configuration to produce longitudinal waves or a radial configuration to produce radial waves, may have a smooth surface or a rough surface. By way of example, the radiation surface 160 may be knurled, spiked, waved, grooved or ribbed. These are only examples of end surfaces and/or radial surfaces of ultrasound tips that can be used with the ultrasound tip 150 according to the present invention. Other surfaces may be similarly effective. Furthermore, any end radiation surface 160 may be mixed and matched with any radial radiation surface 160.

The carrier media 310 may be specifically designed for this application, or existing products may be utilized. The carrier media 310 is preferably portable and easily transported by the user. The carrier media 310 may be reusable or of a disposable design. Embodiments of carrier media 310 include credit/debit cards, adhesive bandage, blank plastic cards or plastic films/webs. The present invention may be combined with an Acu-Node™ pliable pad to provide short-term pain relief to the long-term treatment of chronic pain through the Acu-Node™ system.

The carrier media 310 is preferably of an inexpensive plastic construction particularly in a disposable embodiment. Carrier media materials of construction typically include; PVC (polyvinylchloride), PVCA (polyvinylchloride acetate), LDPE (low density polyethylene), PE and latex rubber.

Various other types of materials may be used to carry the invention such as, but not limited to, crystalline polymers, amorphous polymers, and polymer alloys. A crystalline polymer, meaning a polymer presenting three-dimensional order on the level of atomic dimensions.

Since credit/debit cards are already ubiquitously carried by users they make a convenient option for use as a carrier media 310. A credit card typically has dimensions of 3.375-inches by 2.125-inches with a 30-mil thickness. Common materials of composition include PVC, PCVA, PET and Polyester/PVC blends. The magnetic strip and smart card programmable memory of existing credit cards have been found to be compatible with the activation required for use in some embodiments of this invention.

The Acu-Node™ brand therapeutic pressure pad, used for treatment of chronic pain is also a compatible embodiment for use for immediate topical pain relief with this invention. An Acu-Node™ generally includes a LDPE pad that is approximately the size of a credit card and held in place with velcro and an elastic strap. The LDPE pad generally includes a surface of pins, bumps or ridges that provide the therapeutic effect.

Bandages, of course often include an integral adhesive for direct attachment to a user's skin. The immediate topical pain relief of the present invention, is often a desired feature to cooperate with the wound protection features of a bandage. Furthermore, bandages are typically prepackaged in individual storage containers which may designed to also maintain the activation energy imparted to the bandage.

In a further embodiment, a thin film or web can be energized and then wrapped around the affected area of the user. A number of plastics, including PVC can be manufactured in thicknesses of less than 0.001-inches and still maintain sufficient structural strength to be used as a wrapping around a user. In addition, a film may be prefolded before being energized, energized while in a folded state and unfolded for use. For example, a 0.001-inch thick film could be folded into 30 layers before reaching the thickness of a conventional credit card.

Exposing a carrier media 310 such as a credit card to ultrasonic waves energizes the plastic in the credit card which can then be used to provide pain relief when contacted against the affected area of the user. The energized plastic is applied to a user to provide an analgesic effect either immediately after being energized or the energized polymer can be stored for use at a future time.

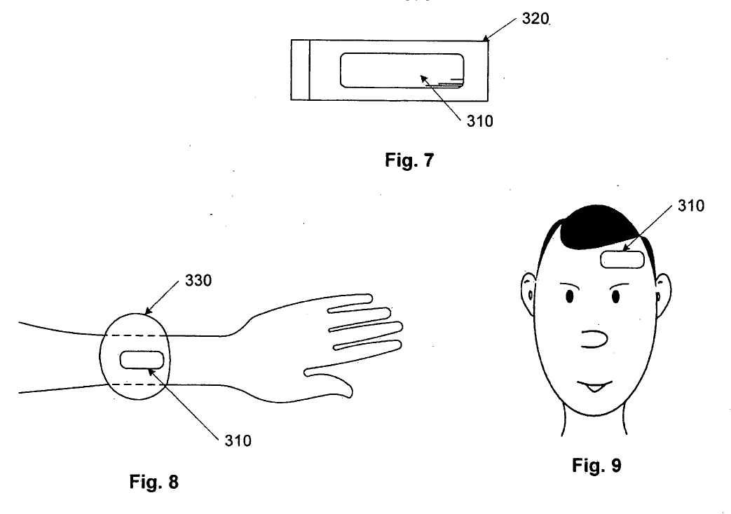

If not used immediately after energizing, the carrier media 310 is preferable placed within a storage container to avoid dissipation of the activation energy. The use of the storage container allows the carrier media 310 to store energy, thus allowing the carrier media 310 to be removed from the storage container at a future time to be placed on a user provide an analgesic effect. The storage container may be a simple bag or envelope constructed of a plastic, such as polyethylene, or a rigid case constructed of a plastic or metal material. FIG. 7 is a perspective view of a carrier media 310 within a storage container 320. If the carrier media 310 is not to be immediately used, then it may be sealed in a plastic encasement, such as a plastic bag.

Opaque materials such as metals, or opaque plastics may retain the energy of the energized carrier media 310 longer than a clear plastic.

Materials of use for the storage containers 320 are selected on the basis of the materials ability to extend the life of the carrier media 310, as well as cost, convenience and ease of use. Examples of storage container material to use include, but are not limited to, plastic bags, plastic sleeves, film, metal or fabric. Other storage materials that extend the effective life of the carrier media 310 may be similarly effective. A selection of a combination of carrier media 310 and storage container 320 that allow for an activation frequency on a weekly basis may be desired as a balance of the users preferred interaction with a kiosk or ATM.

To be self sealing around the carrier media 310, the storage container 320 may consist of at least one adhesive and one non-adhesive side or edge. Alternately, the storage container may have a zip-lock seal, fold over seal, friction fit, or may be sealed by heat, adhesives, or ultrasonic welding.

The carrier media 310 may be simply held in place by the user, or for the user's convenience, a media attachment mechanism may be used. FIG. 8 is a perspective schematic view of a carrier media 310 held in place on a user with a media attachment mechanism. An adhesive bandage would generally include its own adhesive for attachment to the user's skin. Alternatively, an elastic or Velcro strap dimensioned to hold the carrier media 310 or credit card and releasably attached to the user's skin. For example, medical or surgical tape with an adhesive having a paper or polyethylene backing typically used to hold gauze or bandage wounds and similarly may be used to hold the carrier media 310 in place. FIG. 9 is a schematic view of a carrier media 310 held in place on a user's head such as for headache relief. The carrier media 310 may be attached with adhesive, tape or a simple headband for example.

This invention provides a very flexible system to provide convenient access to a pain relief system that is safe, inexpensive, simple to use and does not require administration or control of chemicals or drugs. The carrier media 310 may be made available in a form that is easy to use and provides a very convenient portable device that can be activated at home for routine users, or at convenient locations for occasional users.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive.

Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

I claim:

1. A portable topical pain relief system comprising:
an energizing station for energizing a carrier media using ultrasound energy;
the energizing station having an ultrasound generator driving an ultrasound transducer;
the ultrasound transducer attached to an ultrasound horn;
the ultrasound horn having an ultrasound tip at a distal end of the ultrasound horn; and
at least a portion of the ultrasound tip forming a radiation surface for emitting ultrasound waves into the carrier media.

2. The system of claim 1 wherein the energizing station is incorporated into a kiosk.

3. The system of claim 1 wherein the carrier media is PCV material.

4. The system of claim 1 wherein the carrier media is a credit card.

5. The system of claim 1 wherein the carrier media is reusable.

6. The system of claim 1 including a storage container for the carrier media is placed between being energized and used.

7. The system of claim 6 wherein the storage container consists of a polyethylene envelope.

8. The system of claim 1 wherein the carrier media is a therapeutic pressure pad.

9. The system of claim 8 including an elastic strap for holding the carrier media.

10. The system of claim 1 wherein the ultrasound tip emits ultrasound waves from a radial surface.

11. A portable topical pain relief system comprising:
an energizing station for energizing a carrier media using ultrasound energy;
the energizing station having an ultrasound generator driving a piezo-material;
at least a portion of the piezo-material forming a radiation surface for emitting ultrasound waves into the carrier media.

12. A method for portable topical pain relief comprising:
inserting a carrier media into an energizing station;
positioning the carrier media in close proximity to an ultrasound tip;
delivering ultrasound energy to the carrier media from the ultrasound tip;
removing the carrier media from the energizing station;
applying the carrier media to a users skin to obtain pain relief.

13. The method of claim 11 having the additional step of storing the carrier media within a storage container.

14. The method of claim 11 wherein the carrier media is reusable.

15. The method of claim 11 wherein the carrier media is a PVC material.

16. The method of claim 11 wherein the energizing station is a multi-purpose kiosk.

17. The method of claim 11 wherein the carrier media is a therapeutic pressure pad.

18. The method of claim 16 wherein the carrier media also provides treatment for chronic pain.
